(19) United States
(12) Patent Application Publication
(10) Pub. No.: US 2006/0135952 A1
(43) Pub. Date: Jun. 22, 2006

(54) Corrective intraocular lens and associated methods

(76) Inventors: Eugene O. Curatu, Orlando, FL (US); Richard Ty Olmstead, Oviedo, FL (US)

Correspondence Address: ALCON RESEARCH, LTD., R&D COUNSEL, Q-148, 6201 SOUTH FREEWAY, FORT WORTH, TX, 76134-2099, US

(21) Appl. No.: 11/018,590

(22) Filed: Dec. 21, 2004

(51) Int. Cl. A61F 9/008; A61F 9/013; A61F 2/16

(52) U.S. Cl. 606/5; 623/6.22; 351/246

(57) ABSTRACT

A system for providing improved vision to a patient having undergone an intraocular lens implantation includes a device for measuring an aberration in an eye of a patient having an intraocular lens implanted therein. Computer software is resident on a processor and is adapted to calculate a refraction profile prescription for correcting the measured aberration. An apparatus is also provided for altering a refractive index of a sector of the intraocular lens in situ according to the calculated prescription. The method includes measuring an aberration in an eye of a patient having an intraocular lens implanted therein, calculating a refraction profile prescription for correcting the measured aberration, and altering a refractive index of a sector of the intraocular lens in situ according to the calculated prescription.

### TECHNICAL FIELD OF THE INVENTION

The present invention generally relates to corrective lenses and, in particular, to devices and methods for adjusting an intraocular implant to impart improved visual acuity.

### BACKGROUND OF THE INVENTION

A common problem that affects vision, especially in later life, is the development of cataracts, which cause the natural crystalline lens to become cloudy. A surgical procedure is known to correct cataracts wherein the natural lens is removed and an artificial intraocular lens (IOL) is inserted in its place that replaces the focusing power of the natural lens. Typically the IOL is a homogeneous element comprising a plastic, a hydrogel, or silicone, for example, that may be substantially rigid or foldable for insertion. Bicomposite IOLs are also known that comprise a first material in the optic portion and a second material in the haptic portion.

However, perfect vision is rarely restored after cataract removal, and the patient is typically required to wear glasses to provide one or both of distance and near vision. This can be addressed in some cases with the use of a multifocal lens, an implantable contact lens, or an intracorneal lens implant.

In commonly owned U.S. Pat. No. 6,663,240 is described a method of manufacturing an IOL that has been customized to provide optimum vision for an eye that has previously experienced corneal refractive surgery. One of the embodiments disclosed in the U.S. Pat. No. 6,663,240 patent includes a two-step procedure in which a primary lens is implanted in a first surgery and a second, supplementary lens is implanted in a second surgery following data collection on the results of the implantation of the primary lens.

It would be beneficial to provide an IOL and a method for implanting same that is customizable in situ to provide optimal vision following cataract surgery.

### BRIEF SUMMARY OF THE INVENTION

The present invention, a first aspect of which includes a system for providing improved vision to a patient having undergone an intraocular lens implantation, comprises a device for measuring an aberration in an eye of a patient having an intraocular lens implanted therein. Computer software is resident on a processor and is adapted to calculate an IOL modulation refraction profile prescription for correcting the measured aberration. Means are also provided for altering the refractive profile of a sector of the intraocular lens in situ according to the calculated prescription.

The method of the present invention comprises the steps of measuring an aberration in an eye of a patient having an intraocular lens implanted therein and calculating a refraction profile prescription for correcting the measured aberration. The method also includes the step of altering a refractive index of a localized sector of the intraocular lens in situ according to the calculated prescription.

The features that characterize the invention, both as to organization and method of operation, together with further objects and advantages thereof, will be better understood from the following description used in conjunction with the accompanying drawing. It is to be expressly understood that the drawing is for the purpose of illustration and description and is not intended as a definition of the limits of the invention. These and other objects attained, and advantages offered, by the present invention will become more fully apparent as the description that now follows is read in conjunction with the accompanying drawing.

### BRIEF DESCRIPTION OF THE DRAWING

A more complete understanding of the present invention and the advantages thereof may be acquired by referring to the following description, taken in conjunction with the accompanying drawings in which like reference numbers indicate like features and wherein:

FIG. 1 is a flowchart outlining an embodiment of a method for providing improved vision to a patient;

FIG. 2 is a schematic of a system for providing improved vision to a patient; and

FIG. 3 is a detailed view of an intraocular lens being modified with a laser beam.

### DETAILED DESCRIPTION OF THE PREFERRED EMBODIMENT

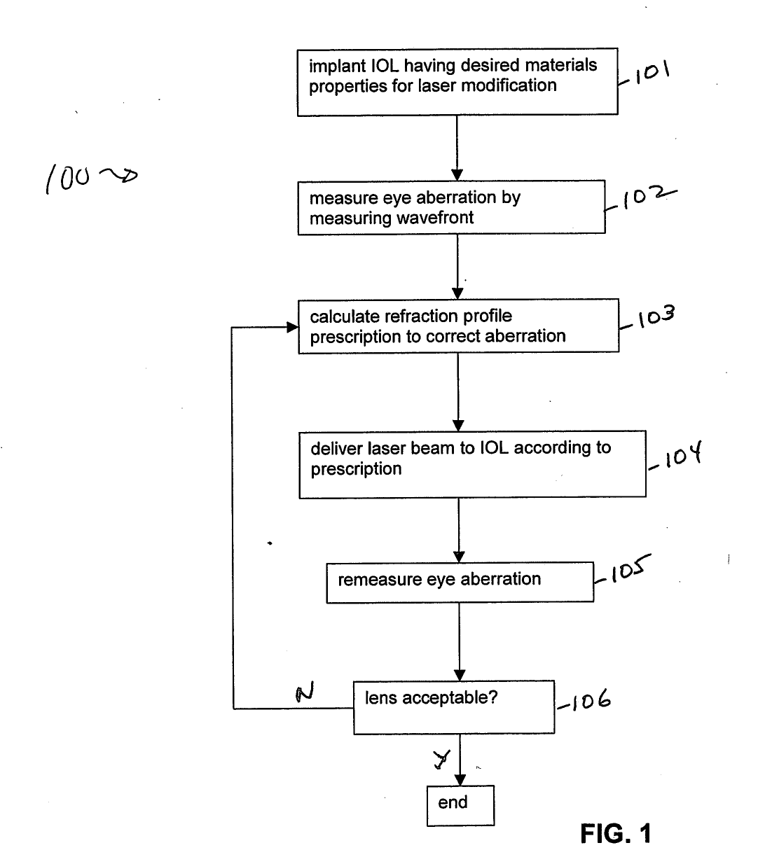

A description of the preferred embodiments of the present invention will now be presented with reference to FIGS. 1-3.

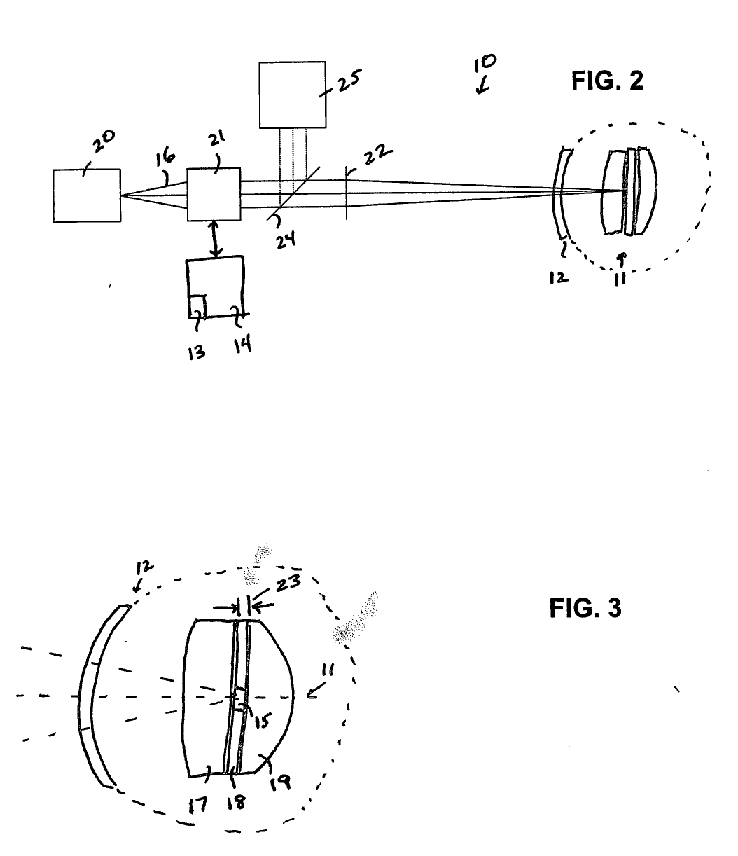

A method 100 (FIG. 1) and system 10 (FIG. 2) are provided for imparting improved vision to a patient who has undergone an intraocular lens (IOL) implantation. Preferably an IOL 11 has been implanted that has specific materials characteristics that will be discussed in the following (block 101). An aberration is measured in an eye 12 of a patient that has had the intraocular lens 11 implanted therein (block 102). Such a measurement may be made, for example, with the use of a Hartmann-Shack wavefront measurement system such as known in the art, including, but not intended to be limited to, commonly owned U.S. Pat. Nos. 5,849,006, 6,261,220, 6,271,914, 6,270,221, 6,578,963, and 6,598,975. Such systems operate, for example, by illuminating a retina of the eye 12 and measuring a wavefront emanating therefrom, and determining a presence of high-order aberrations caused by lens misalignment, i.e., decentration and tilt.

Using the data collected from the wavefront measurement system, computer software 13 resident on a processor 14 is used to calculate a refraction profile prescription for correcting the measured aberration (block 103). Such a calculation may comprise, for example, applying the equation:
δn=W(x,y)/t
where W(x,y) is the measured wavefront aberration, (x,y) are the normalized coordinates, and t is the thickness of intraocular lens sector to be altered.

Next a refractive profile of a sector 15 of the intraocular lens 11 is altered in situ according to the calculated prescription. Such an alteration is preferably made by delivering a laser beam 16 to the intraocular lens sector 15 in such as way as to modify the intraocular lens sector's refractive profile in a desired pattern commensurate with the calculated refractive prescription (block 104). The refractive index of the material changes as a function of laser intensity and exposure time until a saturation value is reached.

The particular IOL 11 for use with the present system 10 and method 100 comprises a material that is susceptible to refractive index alteration by the laser beam 16. In an exemplary embodiment as illustrated in FIG. 3, the IOL 11 comprises a plurality of layers, here three 17-19, proceeding from the outermost layer 17 closest to the eye surface, through a central, beam-susceptible layer 18, to an innermost layer 19.

The central layer 18 may comprise, for example, a substantially transparent material that can be “micromachined” with the use of the laser beam 16. For example, small regions of the central layer 18 can be superheated to cause multiphoton absorption and avalanche ionization, which will effect a refractive index modification in a very small volume. Alternatively, the central layer 18 may comprise a material doped with a molecule susceptible to photochemical modification by the laser beam 16. However the refractive index modification is achieved, the central layer 18 is isolated from the rest of the eye 12 by the enveloping layers 17,19, and thus the eye 12 is protected from any “hot spot” that is induced. An exemplary material for the enveloping layers 17,19 may comprise an acrylic. The central layer 18 may comprise a material such as poly-methyl methacrylate (PMMA), which changes state upon superheating, although this is not intended as a limitation.

In a particular embodiment, the laser beam 16 is delivered from a pulsed, variable-frequency laser 20 that is adapted to micromachine the central layer 18, which then acts as a phase plate. Preferably the laser beam 16 is scanned under the direction of a delivery system 21 that includes a spatial light modulator and other optical elements to achieve the desired refractive index pattern.

A focusing lens 22 is positioned in the beam path upstream of the eye 12. The focusing lens preferably has an F-number (F/#) that provides a depth of focus (dof) substantially matching a thickness t 23 of central layer 18.
dof=2.44λ(F/#)²
which approaches, as dof approaches t,
F/#=(t/2.44×)^{1/2}

In order to monitor the progress of the procedure, a beamsplitter 24 is positioned upstream of the focusing lens 22, directing a portion of the beam 16 to a monitor such as a video camera 25.

Once the refractive-index-changing procedure is complete, preferably the eye 12 should be measured again for any remaining aberration (block 105), in case additional alteration should be performed to the IOL 11 (block 106).

In the foregoing description, certain terms have been used for brevity, clarity, and understanding, but no unnecessary limitations are to be implied therefrom beyond the requirements of the prior art, because such words are used for description purposes herein and are intended to be broadly construed. Moreover, the embodiments of the apparatus illustrated and described herein are by way of example, and the scope of the invention is not limited to the exact details of construction.

Having now described the invention, the construction, the operation and use of preferred embodiments thereof, and the advantageous new and useful results obtained thereby, the new and useful constructions, and reasonable mechanical equivalents thereof obvious to those skilled in the art, are set forth in the appended claims.

What is claimed is:

1. A method for providing improved vision to a patient having undergone an intraocular lens implantation comprising the steps of:
measuring an aberration in an eye of a patient having an intraocular lens implanted therein;
calculating a refraction profile prescription for correcting the measured aberration; and
altering a refractive index of a sector of the intraocular lens in situ according to the calculated prescription.

2. The method recited in claim 1, wherein the aberration measuring step comprises illuminating a retina of the eye and measuring a wavefront emanating therefrom.

3. The method recited in claim 2, wherein the refraction profile prescription calculating step comprises applying the equation:
δn=W(x,y)/t
where W(x,y) is the measured wavefront aberration, (x,y) are the normalized coordinates, and t is the thickness of intraocular lens sector to be altered.

4. The method recited in claim 1, wherein the altering step comprises delivering a laser beam to the intraocular lens sector, the laser beam adapted to modify the intraocular lens sector refractive index in a desired pattern commensurate with the calculated refraction profile prescription.

5. The method recited in claim 4, wherein the laser beam is delivered from a laser adapted to micromachine the intraocular lens.

6. The method recited in claim 5, wherein the laser comprises a variable-frequency laser.

7. The method recited in claim 4, wherein the delivering step comprises scanning the laser beam to achieve the desired refraction profile pattern.

8. The method recited in claim 4, wherein the delivering step comprises directing the laser beam through a focusing lens prior to incidence on the intraocular lens sector.

9. The method recited in claim 4, wherein the intraocular lens comprises a plurality of layers of disparate materials, at least one of the materials susceptible to refractive index alteration by the laser beam.

10. The method recited in claim 9, wherein the intraocular lens comprises three layers, a central layer comprising the susceptible material.

11. The method recited in claim 9, wherein the susceptible material is positioned deeper into the eye than an outermost layer.

12. The method recited in claim 11, wherein the delivering step comprises directing the laser beam through a focusing lens prior to incidence on the intraocular lens sector, the focusing lens having an F-number (F/#) provides a depth of focus (dof) substantially matching a thickness of susceptible material layer.

13. The method recited in claim 4, wherein the altering step comprises superheating the intraocular lens sector to a temperature sufficient to change refractive properties thereof.

14. The method recited in claim 4, wherein the intraocular lens sector comprises a material doped with a molecule susceptible to photochemical modification, and the altering step comprises delivering a laser beam adapted to achieve the photochemical modification of the doping molecule.

15. The method recited in claim 4, further comprising the step of splitting the laser beam upstream of the eye and directing the split-off beam to a monitoring device.

16. The method recited in claim 4, wherein the delivering step comprises modifying the laser beam with the use of a spatial light modulator to achieve the desired refractive pattern.

17. The method recited in claim 1, further comprising the step of measuring an aberration in the patient eye following the refractive index altering step.

18. A system for providing improved vision to a patient having undergone an intraocular lens implantation comprising:
a device for measuring an aberration in an eye of a patient having an intraocular lens implanted therein;
computer software installable on a processor for calculating a refraction profile prescription for correcting the measured aberration; and
means for altering a refractive index of a sector of the intraocular lens in situ according to the calculated prescription.

19. The system recited in claim 18, wherein the aberration measuring device comprises means for illuminating a retina of the eye and for measuring a wavefront emanating therefrom.

20. The system recited in claim 19, wherein the refraction profile prescription calculating software comprises a code segment for applying the equation:
δn=W(x,y)/t
where W(x,y) is the measured wavefront aberration, (x,y) are the normalized coordinates, and t is the thickness of intraocular lens sector to be altered.

21. The system recited in claim 18, wherein the altering means comprises means for delivering a laser beam to the intraocular lens sector, the laser beam adapted to modify the intraocular lens sector refractive index in a desired pattern commensurate with the calculated refraction profile prescription.

22. The system recited in claim 21, wherein the laser beam delivering means comprises a laser adapted to micromachine the intraocular lens.

23. The system recited in claim 22, wherein the laser comprises a variable-frequency laser.

24. The system recited in claim 21, wherein the laser beam delivering means comprises a scanner for scanning the laser beam to achieve the desired refractive index pattern.

25. The system recited in claim 21, wherein the laser beam delivering means comprises a focusing lens and means for directing the laser beam through the focusing lens upstream of the intraocular lens sector.

26. The system recited in claim 21, wherein the intraocular lens comprises a plurality of layers of disparate materials, at least one of the materials susceptible to refractive index alteration by the laser beam.

27. The system recited in claim 26, wherein the intraocular lens comprises three layers, a central layer comprising the susceptible material.

28. The system recited in claim 26, wherein the susceptible material is positioned deeper into the eye than an outermost layer.

29. The system recited in claim 28, wherein the laser beam delivering means comprises a focusing lens and means for directing the laser beam through the focusing lens upstream of the intraocular lens sector, the focusing lens having an F-number (F/#) provides a depth of focus (dof) substantially matching a thickness of susceptible material layer.

30. The system recited in claim 21, wherein the laser beam is adapted to superheat the intraocular lens sector to a temperature sufficient to change the refractive index thereof.

31. The system recited in claim 21, wherein the intraocular lens sector comprises a material doped with a molecule susceptible to photochemical modification, and the laser beam is adapted to achieve the photochemical modification of the doping molecule.

32. The system recited in claim 21, further comprising a monitoring device and a beam splitter for splitting the laser beam upstream of the eye and directing the split-off beam to the monitoring device.

33. The system recited in claim 21, wherein the laser beam delivering means comprises a spatial light modulator for modifying the laser beam to achieve the desired refractive index pattern.
